(19) United States
(12) Patent Application Publication
(10) Pub. No.: US 2013/0261747 A1
(43) Pub. Date: Oct. 3, 2013

(54) ALIF Spinal Implant

(71) Applicant: Christophe Geisert, Newtonville, MA (US)

(72) Inventor: Christophe Geisert, Newtonville, MA (US)

(21) Appl. No.: 13/853,417

(22) Filed: Mar. 29, 2013

Related U.S. Application Data
(60) Provisional application No. 61/618,234, filed on Mar. 30, 2012.

(51) Int. Cl. A61F 2/44

(52) U.S. Cl. CPC A61F 2/442
USPC 623/17.16

(57) ABSTRACT

Spinal fusion appliances, delivery devices and methods of using the same are provided. In various embodiments, the appliances include a housing and a flexible tape with opposite ends that are optionally adjustable using an actuator within the housing.

### CROSS-REFERENCE TO RELATED APPLICATION

This application claims priority to U.S. Provisional Application No. 61/618,234, filed Mar. 30, 2012 by Geisert entitled “ALIF Spinal Implant.” The entire disclosure of this application is hereby incorporated by reference for all purposes.

### FIELD OF THE INVENTION

The technology disclosed herein relates, in general, to medical implants and, more specifically, to methods and devices for intervertebral fixation.

### BACKGROUND

Back pain, particularly lower back pain, is the fifth most common reason for all physician visits in the United States. (See Roger Chou et al. Diagnosis and Treatment of Low Back Pain: A Joint Clinical Practice Guideline from the American College of Physicians and the American Pain Society. 147 Annals of Internal Medicine pp. 478-491 (Oct. 2, 2007)) While back pain may be managed in some patients with conservative treatments such as exercise, acupuncture, massage or steroid injection, other patients' pain may be caused by disk conditions such as disk herniation or degeneration, or vertebral conditions such as fracture and spondylolisthesis, which require more invasive treatments such as spinal fusion and fixation.

Spinal fusion is a surgical procedure in which two or more vertebrae are fused to prevent or decrease movement at or around a site of back pain. The fusion of vertebrae is augmented by the use of rigid implantable fixation devices such as bone screws or plates, which limit the movement of vertebrae relative to one another. These devices may be implanted between individual vertebrae in the space usually occupied by the spinal disk, in what is termed “interbody fixation.” Many interbody fixation devices, such as screws and plates, are fully rigid, and prevent movement between fused vertebrae, which may result in decreased patient mobility after the procedure. Other interbody fixation devices incorporate a hinge or a surface that permits limited movement of fused vertebrae relative to one another, but these devices generally apply outward pressure on the fused vertebrae, which may propagate through the spine.

Interbody fixation implants are typically implanted using anterior or posterior approaches. In a posterior approach (a “posterior lumbar interbody fixation” or “PLIF”) the spine is accessed via a posterior incision which is relatively straightforward for the surgeon, but which may result in pain for the patient. In an anterior approach (“ALIF”), the approach is through an abdominal incision, which is more complicated for the surgeon but which may be less painful. Other approaches, such as transforaminal (TLIF) and “extreme lateral” (XLIF) utilize a lateral or posterolateral access.

Interbody fixation implants typically include screws or other means by which they are secured to vertebrae. These means may be angled relative to the central axis of the implant, necessitating the use of a wider surgical access and potentially resulting in increased pain and recovery time for patients. Given the fact that the average cost of operating room time in the US is $15-$25 per minute, there is a constant need in the art to minimize the complexity, and therefore the time required, to perform a surgical interbody fixation procedure. (Stahl, J et al, Reorganizing patient care and workflow in the operating room: a cost-effectiveness study, Surgery, 139:717-728, 2006.)

### SUMMARY OF THE INVENTION

The current invention, in its various embodiments, addresses needs in the art by providing systems and methods for simplified vertebral fixation employing ALIF, PLIF, TLIF and/or XLIF approaches utilizing a narrow surgical access. In one aspect, the invention relates to a medical device that includes a housing, a flexible tape with first and second ends passing through the housing, and an actuator that contacts the flexible tape and adjusts tension on the flexible tape to urge the first and second ends toward one another. The actuator may be a cylindrical body with an aperture through which the tape passes, and the cylindrical body may rotate within the housing, and can include a portion that can be mated with a tool so a user can rotate the actuator within the housing. The medical device can include portions that anchor the first and second ends of the tape within the bone, which portions are optionally planar and include one or more perforations, bumps, or ridges. In addition, the housing can include one or more flanged extensions that are connectable to a bone cage, and flexible tape can include a strengthening element to prevent deformation.

In another aspect, the invention relates to a system for treating a patient that includes a medical device as described above, along with a delivery device that includes multiple bone chisels that define a space sized and/or shaped to fit the medical device. The space in the delivery device may separate the first and second ends by a distance sufficient to permit their being anchored in the cancellous bone of adjacent vertebrae while the housing rests between the vertebrae.

In still another aspect, the invention relates to a delivery device for a spinal fusion appliance that includes multiple bone chisels that, together, define a space sized and/or shaped to accommodate the spinal fusion appliance. The space is optionally shaped so that, when the device is used, each of the first and second ends can be anchored within the cancellous bone of a first vertebra and a second vertebra while the the housing rests between the first and second vertebra

In yet another aspect, the invention relates to a method of treating a patient needing interbody fixation that includes providing a medical device that includes a housing, a flexible tape with first and second ends passing through the housing, and an actuator that contacts the flexible tape and adjusts tension on the flexible tape to urge the first and second ends toward one another, positioning the medical device in relation to two vertebrae so that the first and second ends of the tape are positioned in the cancellous bone of one vertebra or the other while the housing is positioned between the vertebrae, and moving the actuator to urge the first and second ends of the tape toward one another, thereby urging the two vertebrae toward the housing.

### DRAWINGS

In the drawings, like reference characters refer to like features throughout the different views. The drawings are not necessarily to scale, with emphasis being placed on illustration of the principles of the invention

FIG. 1 shows multiple schematic perspective and exploded views of an exemplary interbody fixation appliance according to the invention.

FIG. 2 shows a schematic side view of a portion of an exemplary interbody fixation appliance including a view of the rotating coupling including the central rotating members, the flexible tape, and the vertebral securement features.

FIG. 3 shows multiple schematic perspective views of an exemplary interbody fixation appliance including x-ray views showing the rotating coupling.

FIG. 4 shows a schematic ventral x-ray view of an exemplary interbody fixation appliance deployed between vertebrae, including the positioning of the vertebral securement features and the flexible tape within the vertebrae.

FIG. 5 shows a schematic ventral view as in FIG. 4.

FIG. 6 shows schematic ventral and lateral X-ray views of a spinal column, the lateral view including an exemplary interbody fixation appliance.

FIG. 7 shows a schematic X-ray view of a spinal column.

FIG. 8 shows a schematic lateral X-ray view of adjacent vertebrae with an exemplary interbody fixation appliance disposed therebetween.

FIG. 9 shows a schematic oblique view of an exemplary interbody fixation appliance.

FIG. 10 includes schematic lateral and oblique views of an exemplary interbody fixation appliance including a vertebra into which a vertebral securement feature and a portion of a flexible tape are positioned.

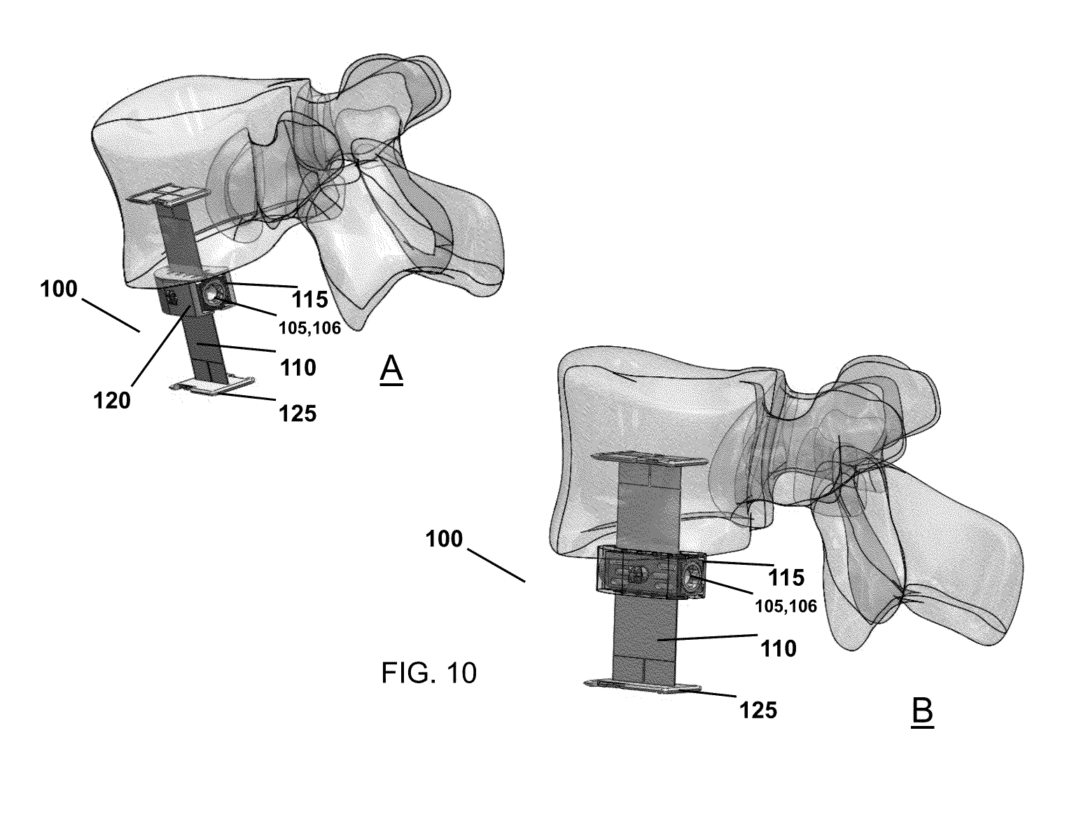

FIG. 11 shows a schematic oblique view of an interbody fixation appliance disposed within two adjacent vertebrae.

FIG. 12 shows multiple views of a delivery device for an interbody fixation appliance, including a close-up view of a head of the delivery device containing an interbody fixation appliance, and a driver for rotating the rotating member, thereby retracting and/or tensioning the flexible member.

FIG. 13 shows schematic views of the positioning of an interbody fixation appliance in TLIF and XLIF procedures.

FIG. 14 shows schematic x-ray oblique views of an interbody fixation appliance in use, illustrating the shortening of portion of the flexible tape extending from either side of the appliance to urge the vertebrae to be fused toward one another.

FIG. 15 shows schematic cutaway views of an exemplary interbody fixation appliance at various stages of retraction of the flexible tape.

FIG. 16 shows a schematic exploded view of an interbody fixation appliance including multiple bone cage portions that can be attached to the main housing.

FIG. 17 shows schematic views of exemplary interbody fixation appliances having curved edges, tapes, and surfaces to better fit in spaces between vertebrae while minimizing trauma.

FIG. 18 shows schematic views of another exemplary interbody fixation appliance for lateral interbody fusion procedures including bone cage portions having curved surfaces.

FIG. 19 shows a schematic x-ray view of a laterial interbody fixation appliance according to FIG. 18 when deployed. It will be appreciated that the drive portion of the central rotating member faces laterally, rather than anteriorly or posteriorly.

FIG. 20 shows multiple schematic views of a locking plate for locking the central rotating member, and the flexible tape, in position.

FIG. 21 shows multiple schematic views of belt fixing systems utilizing ratchet and pawl mechanisms.

FIG. 22 shows an exemplary interbody fusion appliance for use in ALIF procedures, which includes a cam-lobe tensioning system and which utilizes rigid arms rather than a flexible tape.

FIG. 23 shows the multiple schematic views of an interbody fusion appliance as in FIG. 22 as the rigid arms are retracted.

FIG. 24 shows an exemplary interbody fusion appliance that includes a flexible tape comprising a woven textile belt.

### DETAILED DESCRIPTION OF THE PREFERRED EMBODIMENTS

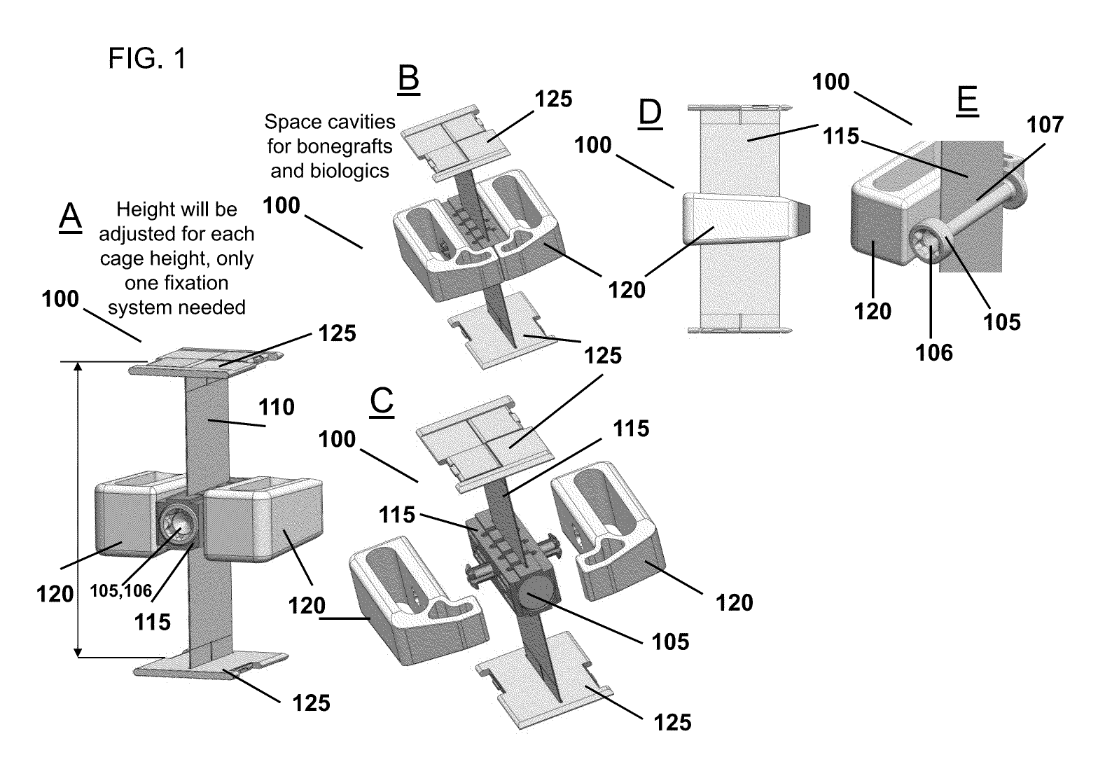

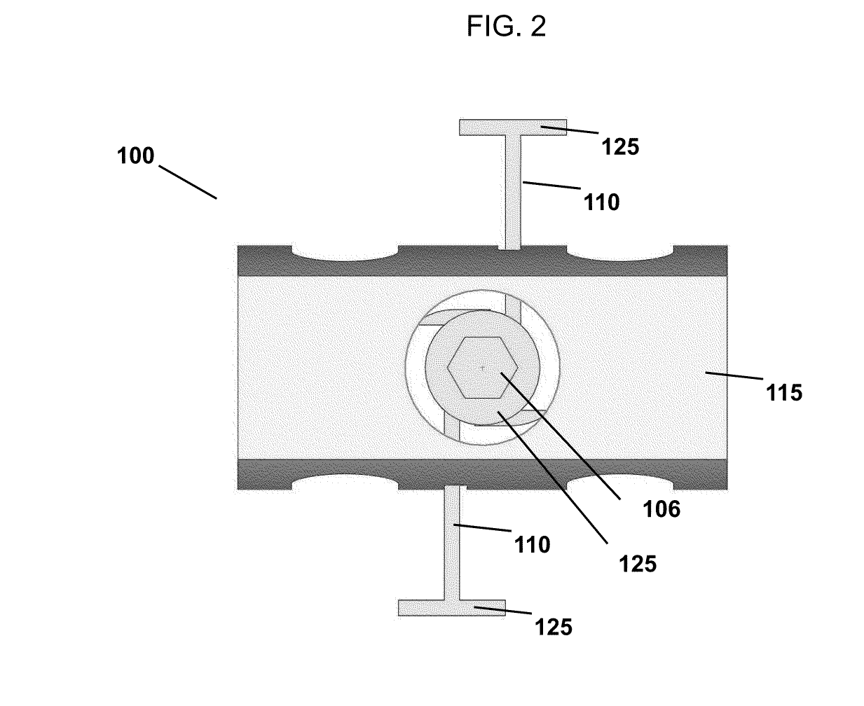

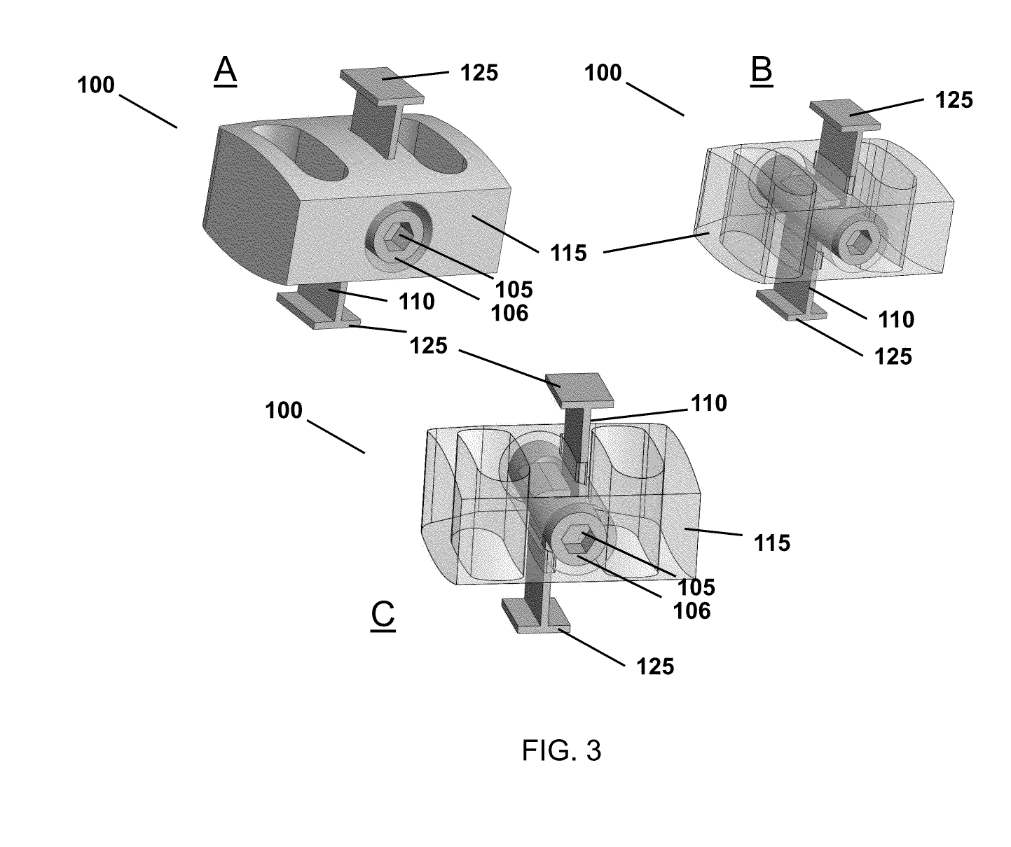

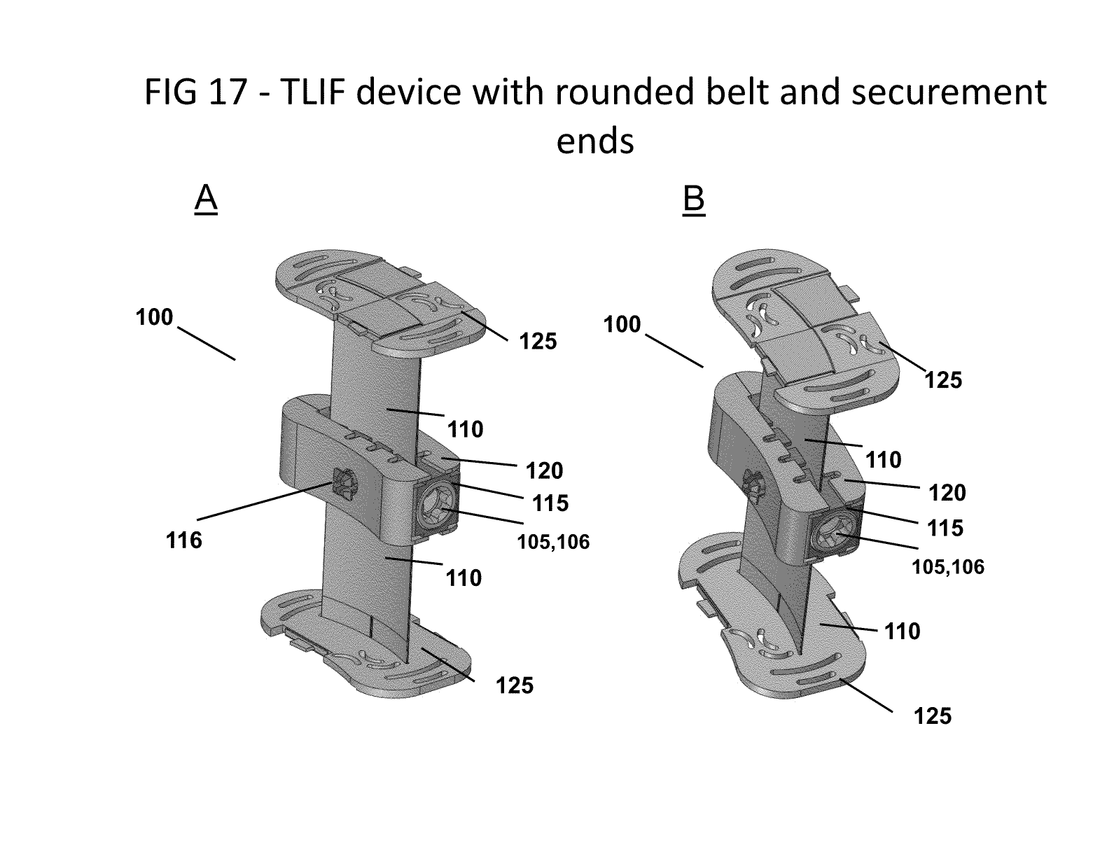

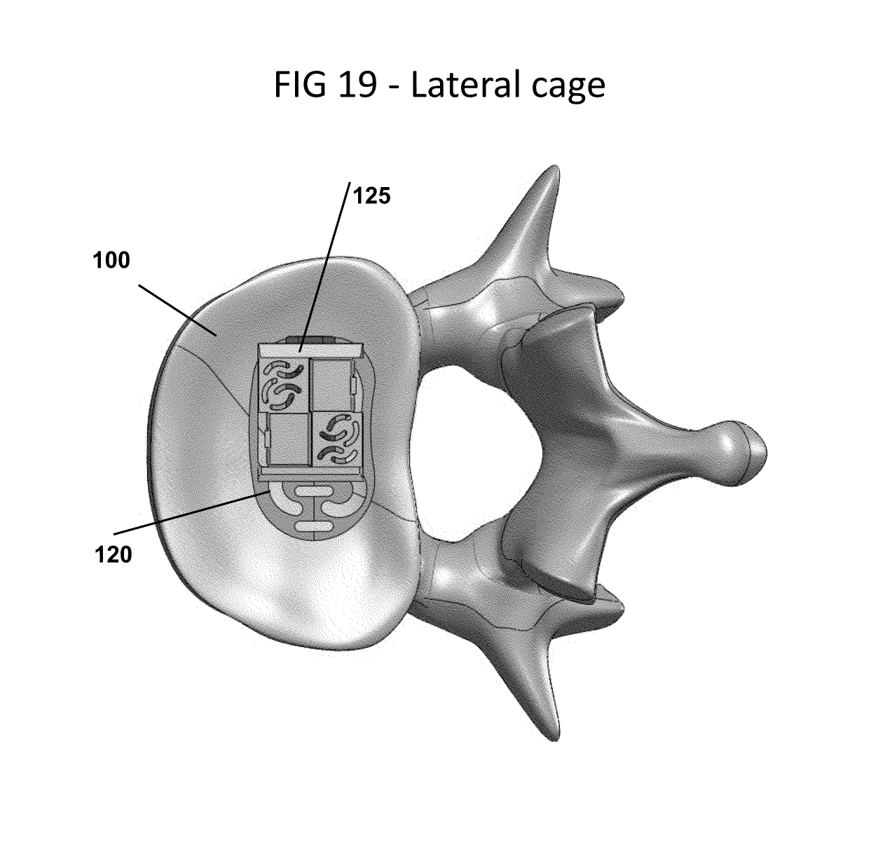

With reference to FIGS. 1-3, an exemplary Interbody fixation appliance according to the invention includes, in some embodiments, an interbody fixation appliance 100 having a central rotating member 105 with a slit 107 therethrough and a flexible tape 110 extending through the slit. The central rotating member 105 is rotatably disposed within a housing 115 that is sized to fit within a space between vertebrae. The housing 115 may include one or more ratcheting features to facilitate the incremental rotation of the rotating member in one or more directions. The housing may also include tabbed projections 116 permitting the permanent or reversible attachment of one or more cages 120, which cages may also be sized to fit within a space between vertebrae and which may include features that promote bone ingrowth such as cavities, pores, holes, and bioactive materials disposed within or coating the cages. The tape 110 has two termini, and vertebral securement features 125 are disposed at the termini. The vertebral securement features 125, in various embodiments, extend away from the tape at an angle, to apply pressure over an area of the vertebrae when tension is applied to the tape and to prevent slippage of the tape. While the figures depict the vertebral securement features 125 extending substantially perpendicularly from the tape, the members may extend away at any suitable angle, including, without limitation, 15°, 30°, 45°, 60°, etc. The vertebral securement features 125 may also include features to promote bone ingrowth such as those listed above. FIG. 17-20 show a vertebral securement feature 125 that includes irregular cut-out portions. Not wishing to be bound to any particular theory, it is believed that by providing voids or other features into which cells can migrate, tissue ingrowth into the vertebral securement features is improved, which in turn improves the quality of fusion achieved by the Interbody fixation appliance.

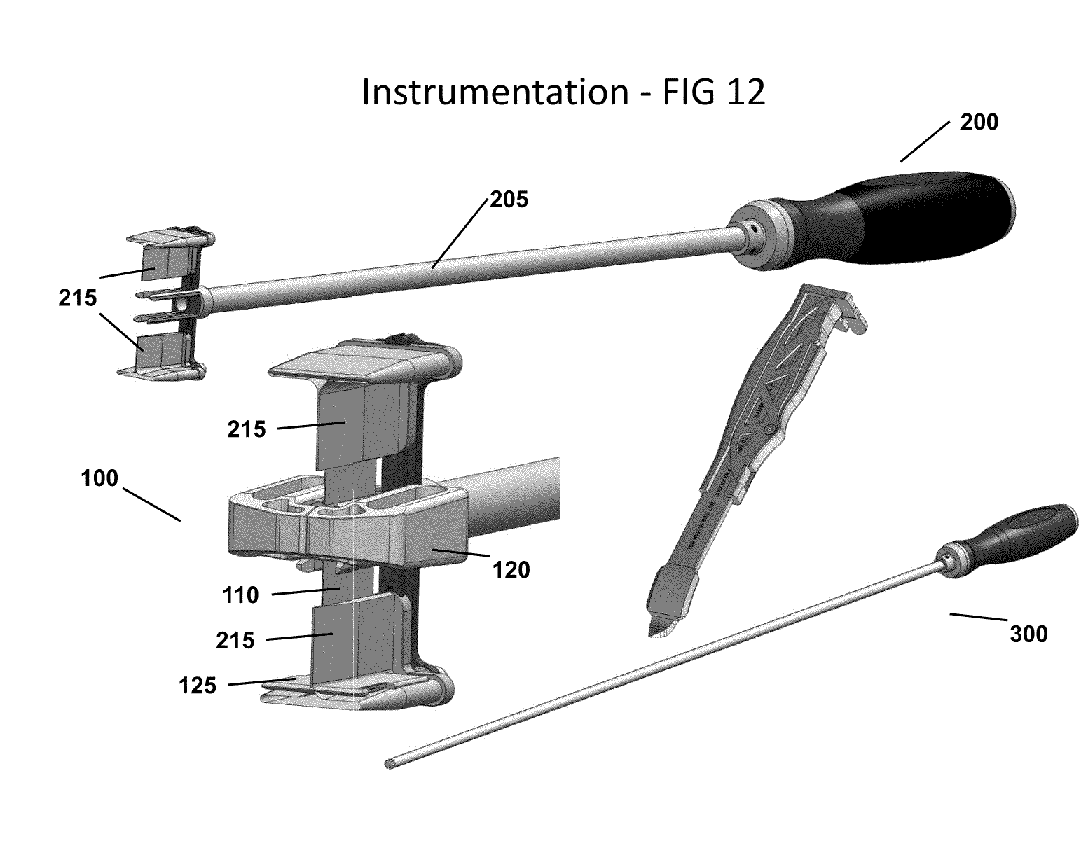

The central rotating element 105, in some embodiments, includes a drive 106 sized to accommodate the tip 305 of a driver 300. The drive may have any suitable shape, including a slot, Philips head, square, hexagonal, octagonal, star, etc. to engage the driver 300. The driver 300, in turn, includes a tip 305 having a shape complementary to the drive 106, so that the driver 300 can be coupled to the drive 106 and used to rotate the central rotating element 105. An exemplary driver is shown in FIG. 12

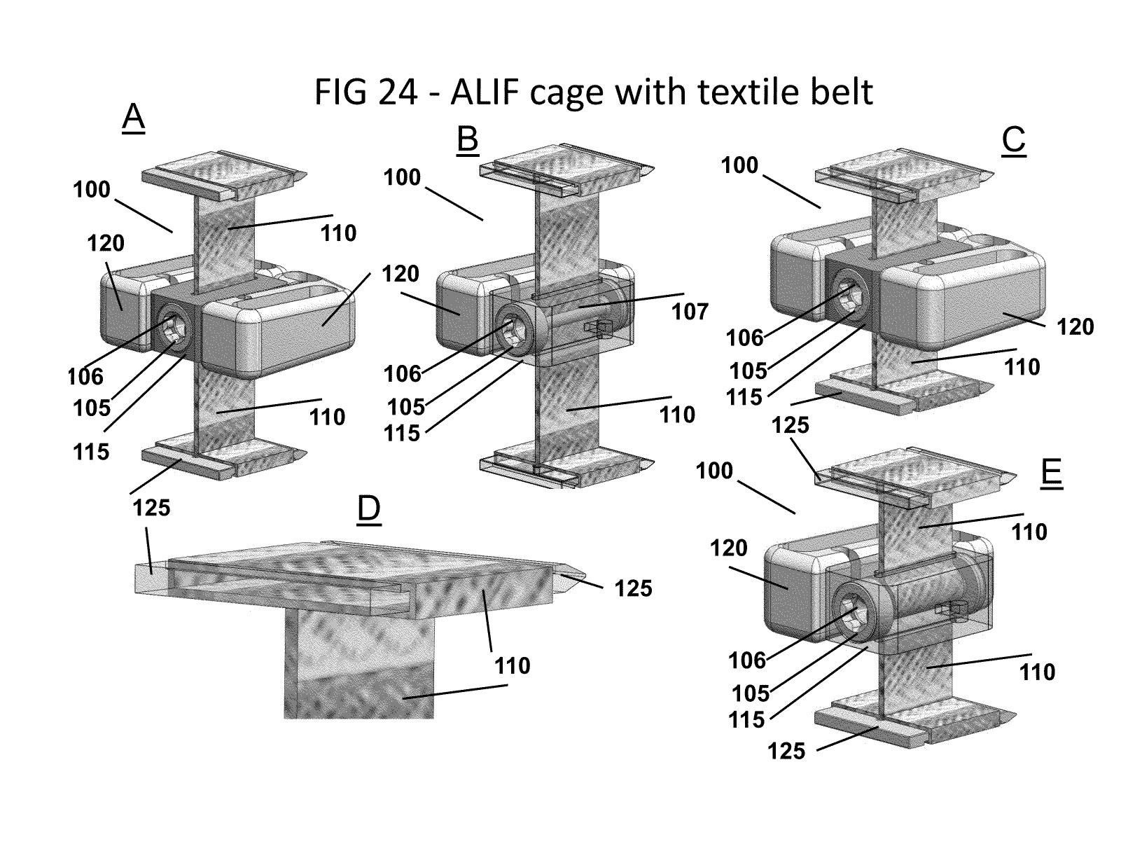

The tape 110 may be made of any suitable material, including metal, plastic, polymer, textile, mesh, etc., and may include features to promote bone ingrowth such as those listed above. A depiction of an exemplary Interbody fixation appliance 100 that includes a textile tape 110 is shown in FIG. 24. The textile tape includes multiple woven fibers, which define surfaces and voids that promote tissue ingrowth, thereby improving the quality of fusion achieved by the Interbody fixation appliance.

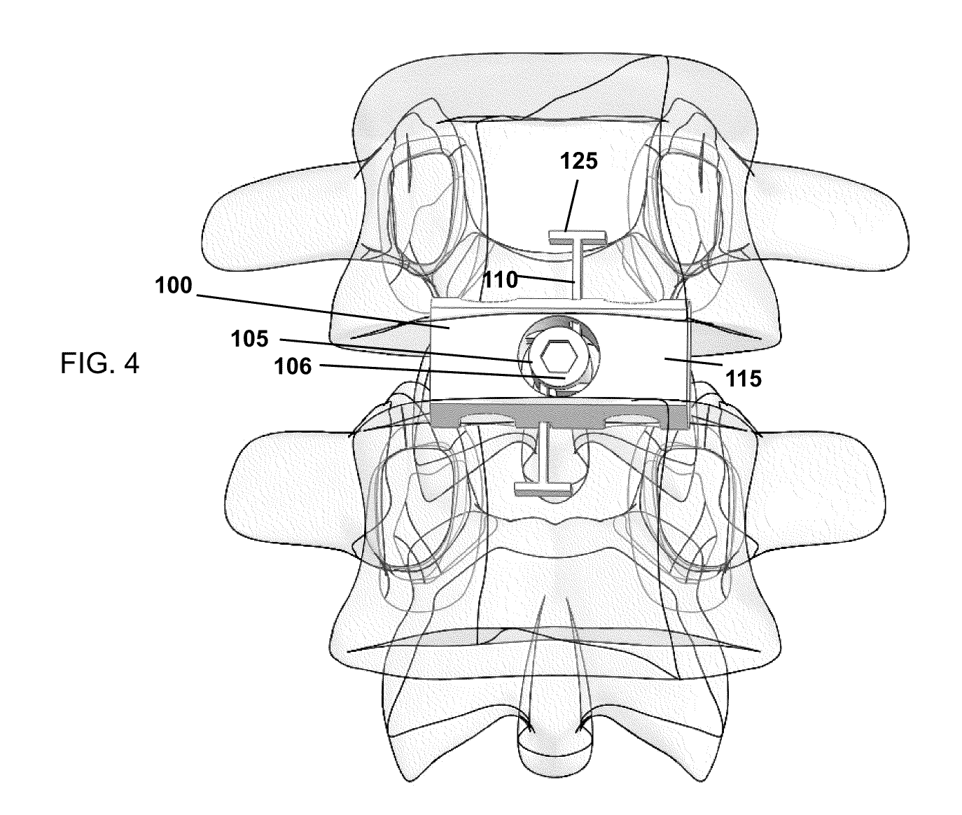

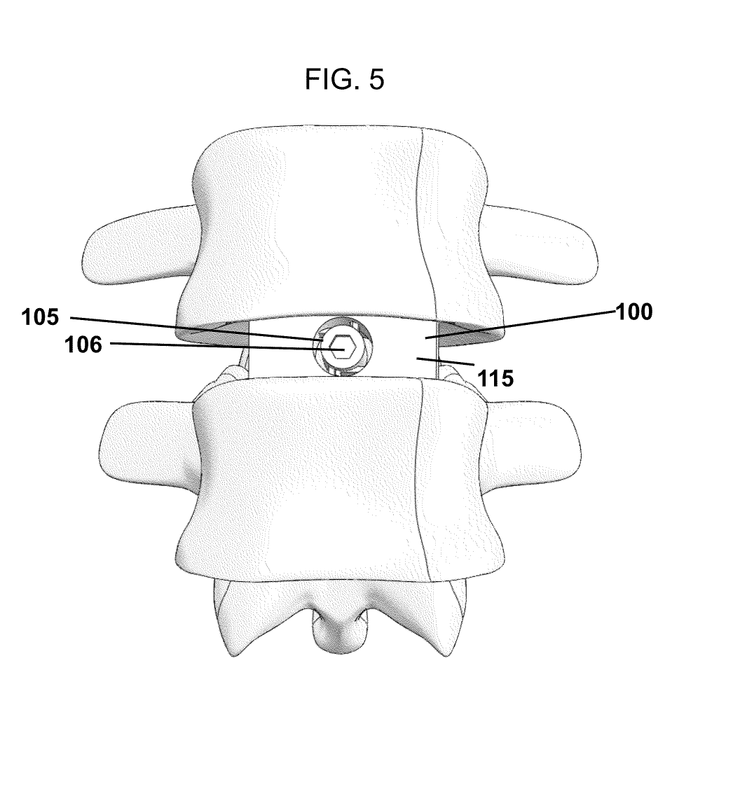

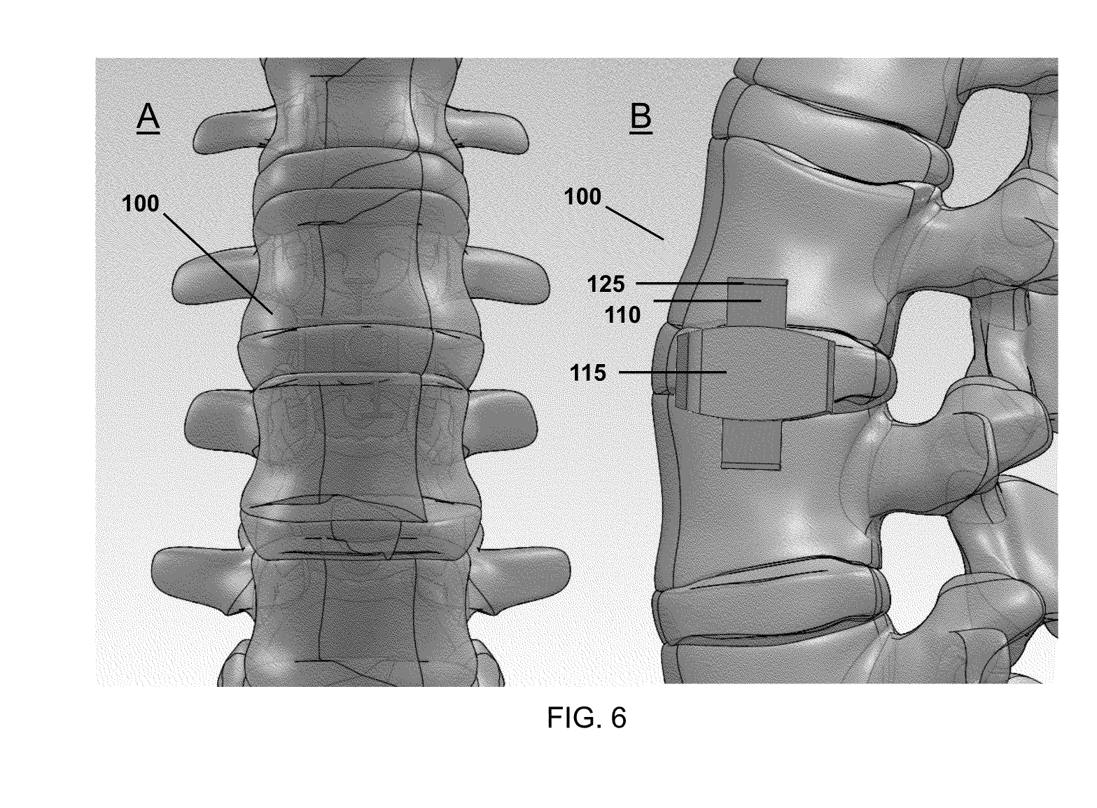

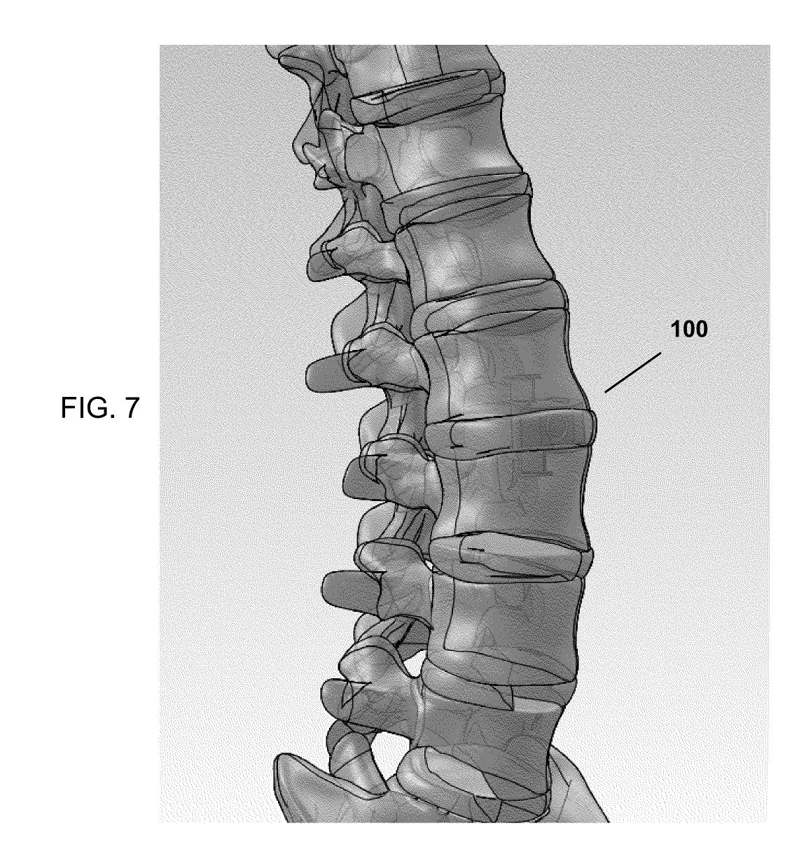

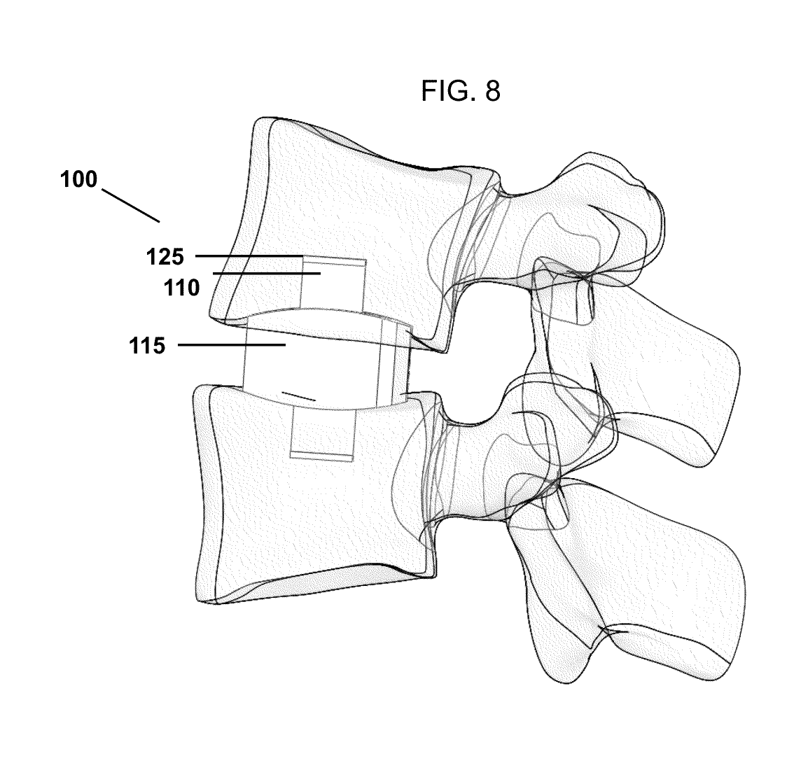

In use, the housing 115 and one or more cages 120 attached thereto are positioned between adjacent vertebrae, as shown in FIGS. 4-8. The tape 110 is fully extended, and the vertebral securement features are disposed on or in the vertebrae. The central rotating member 105 is rotated, applying tension to the tape and urging its termini and the vertebral securement features 125 together.

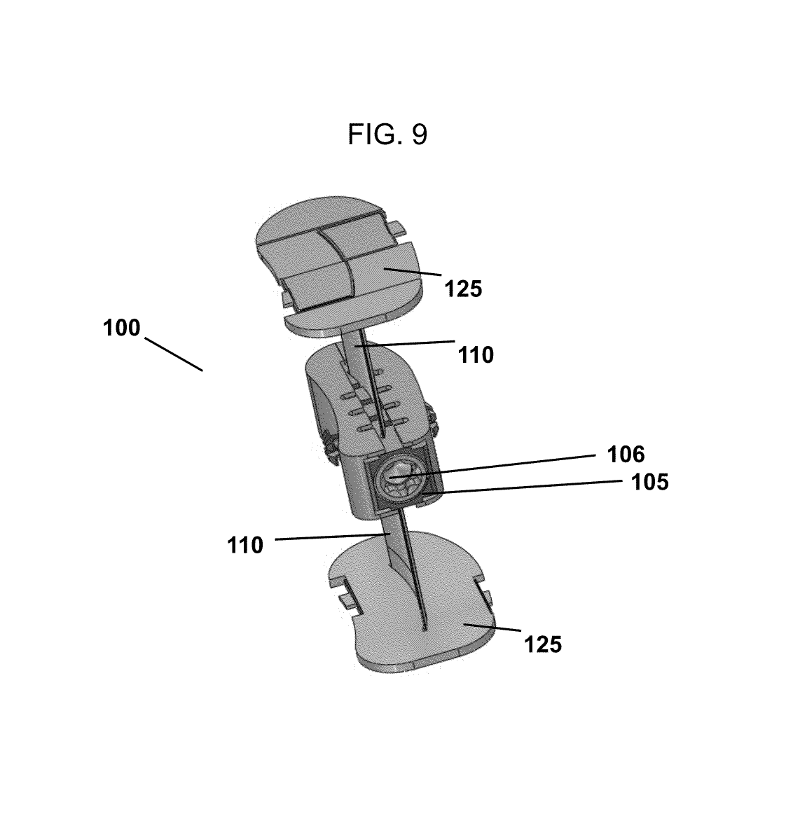

As shown in FIG. 9, the interbody fixation appliance 100 may have any suitable shape, and may incorporate curved features such as housing 115, vertebral securement features 125, and tape 110.

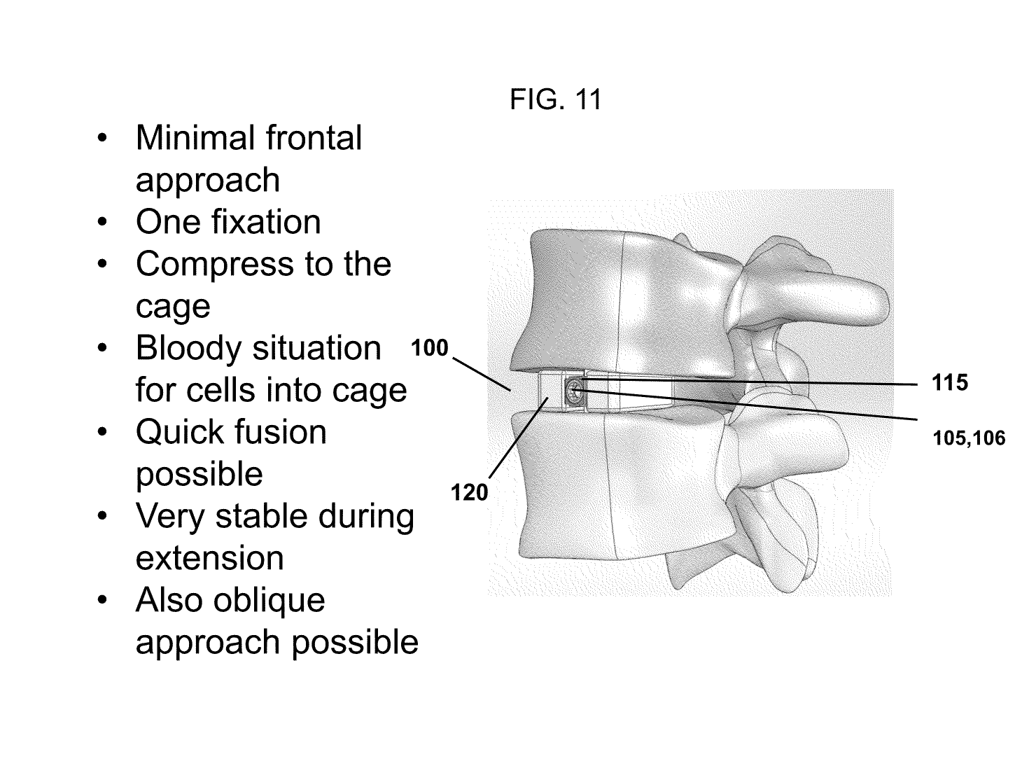

FIG. 11 shows an interbody fixation appliance 100 according to an embodiment of the invention which is adapted for use in ALIF procedures. In an ALIF procedure, the housing 115 and the cage(s) 120 are shaped to be inserted between vertebrae such that the drive 106 of the central rotating member 105 faces the abdomen. In other embodiments, the appliance 100 can be sized for use in PLIF, TLIF or XLIF procedures, as shown in FIGS. 17-19.

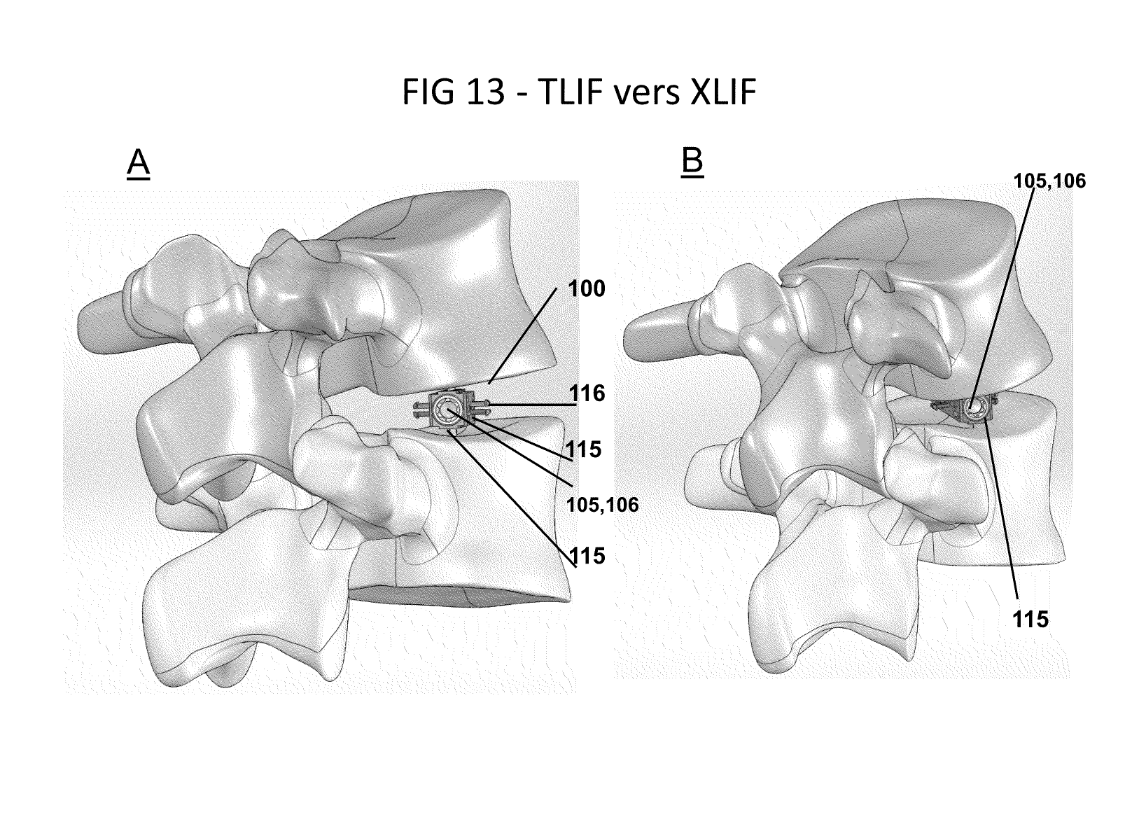

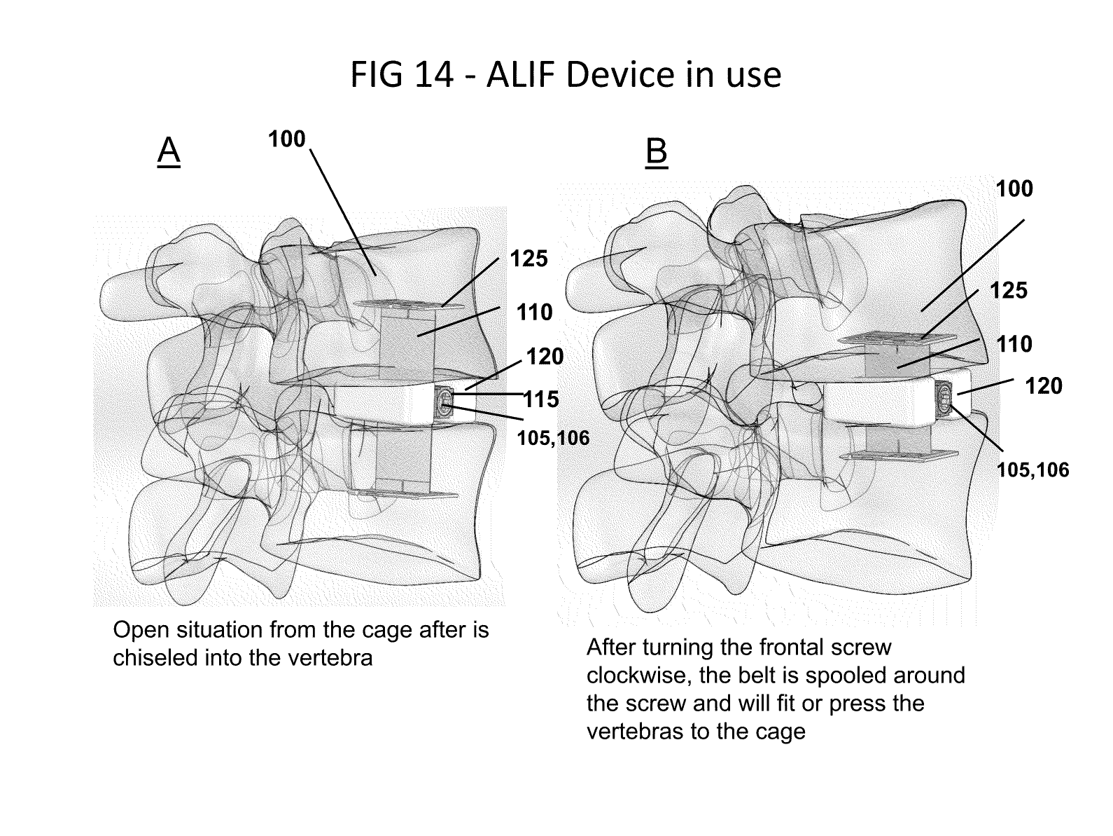

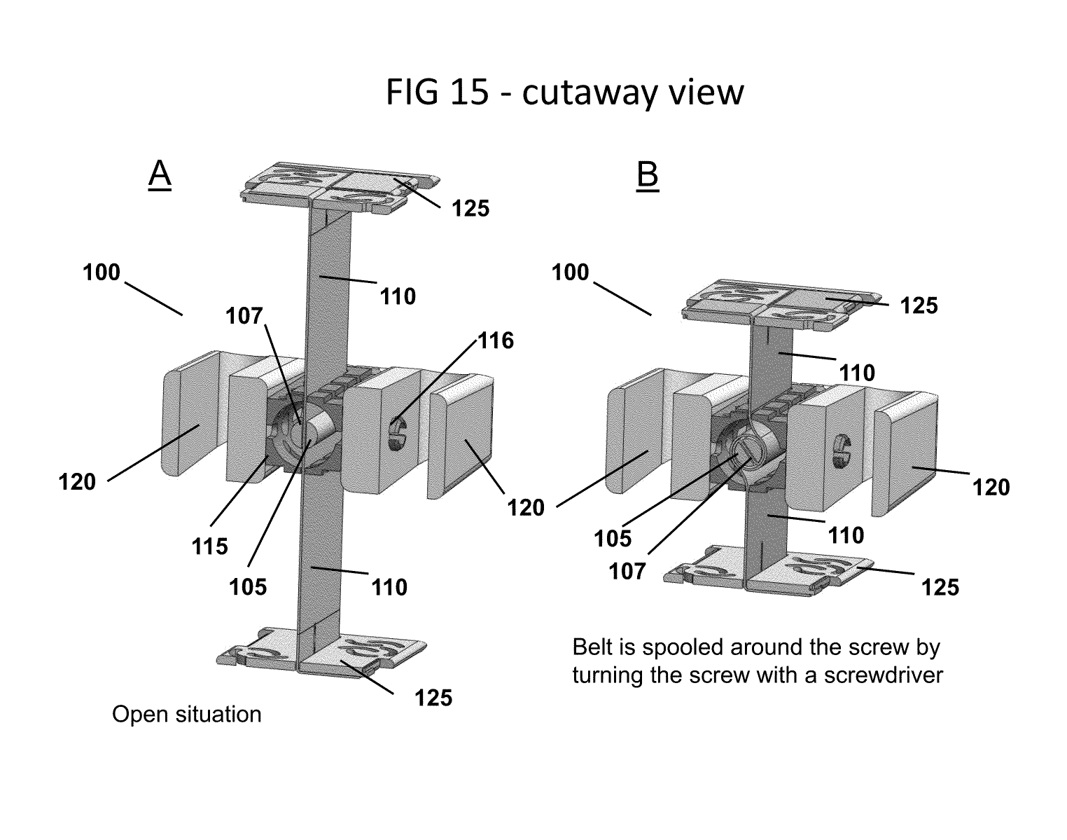

FIG. 12 shows a delivery device (top and inset, left) that may be used to position the interbody fixation appliance 100. The delivery device 200 includes a shaft 205 and an appliance engagement portion 210 sized to engage the interbody fixation appliance 100 with the tape 110 at least partially extended and the vertebral securement features 125 disposed at a distance from the housing 115. The appliance engagement portion 210 includes two chisel portions 215, which include a central recess sized to accommodate an end of the tape 110 and the vertebral securement feature 125. Prior to use, the appliance engagement portion 210 of the delivery device 200 is optionally loaded with an interbody fixation appliance 100; alternatively, the appliance engagement portion 210 is left empty, so that it can create a space or void for subsequent insertion of an appliance 100. In use, the engagement portion 210 is inserted through a surgical access and placed in contact with the anterior surfaces of the vertebrae adjacent to the site at which the appliance 100 will be implanted (in an ALIF procedure). A user applies force along the shaft 205, urging the engagement portion toward the vertebrae of the patient, so that the chisel portions 215 penetrate the vertebrae, positioning appliance 100 such that the housing 115 rests between the vertebrae and the vertebral securement features 125 and tape 110 lie within the vertebrae; alternatively, if the delivery device 200 is not pre-loaded with an appliance 100, use of the delivery device 200 creates a space into which the appliance 100 can be inserted, as above. After the appliance 100 is positioned, the delivery device 200 is removed, and the central rotating member 105 is rotated using a driver 300 having a tip complementary to the drive 106 of the central rotating member 105, thereby spooling the tape 110 about the central rotating member 105 and, consequently, urging the vertebral securement features 125 toward one another, as illustrated in FIG. 14. Spooling of the tape 110 about the central rotating member 105 is further illustrated in the cutaway views of FIG. 15. Those skilled in the art will appreciate that the delivery device 200 and the procedure described above can be adapted for TLIF, XLIF and PLIF procedures, and that the appliance 100 is capable of functioning in different orientations, as shown in FIGS. 13a-b.

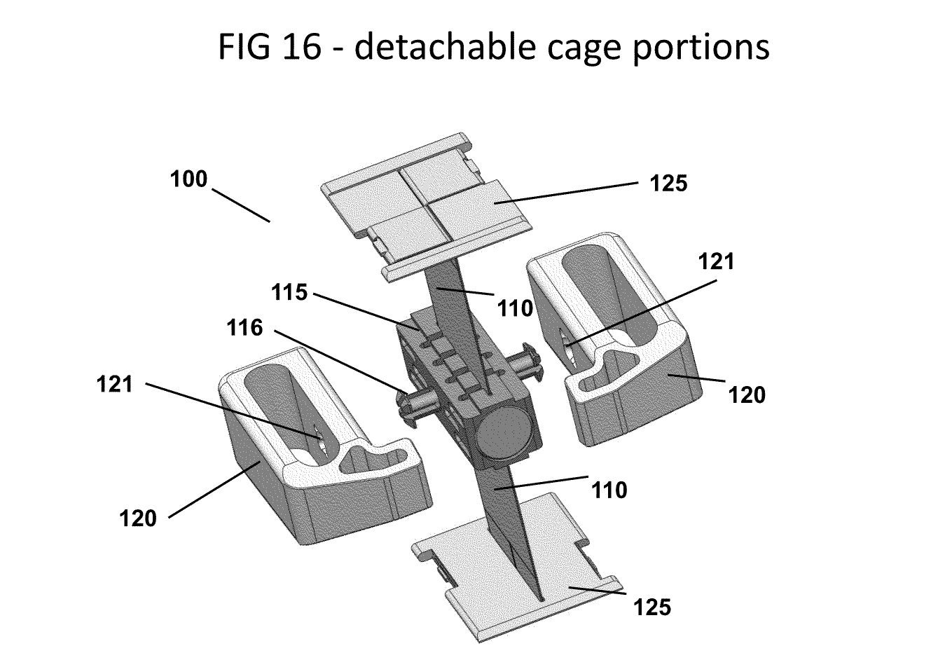

FIG. 16 depicts an appliance 100 having an extended tape 110 in relation to a pair of cages 120 that are attachable to the housing 115 by means of complementary tabbed projections 116 on the housing 115 and holes 121 sized to fit the tabbed projections, thereby achieving a mechanical fit. Appliances according to FIG. 16 which include simple mechanical means for attaching a cage 120 to the housing advantageously permit the cages 120 to be attached to the housing 115 by untrained users without the need for specialized tools or adhesive means, permitting the cages to be attached, for example, en suite immediately prior to implantation of the appliance 100 within a spine or even after the appliance 100 has been implanted. In other embodiments, cages 120 are secured to the housing 115 using means generally known in the art, including adhesive means, heat welding, sonic welding, friction fit, etc.

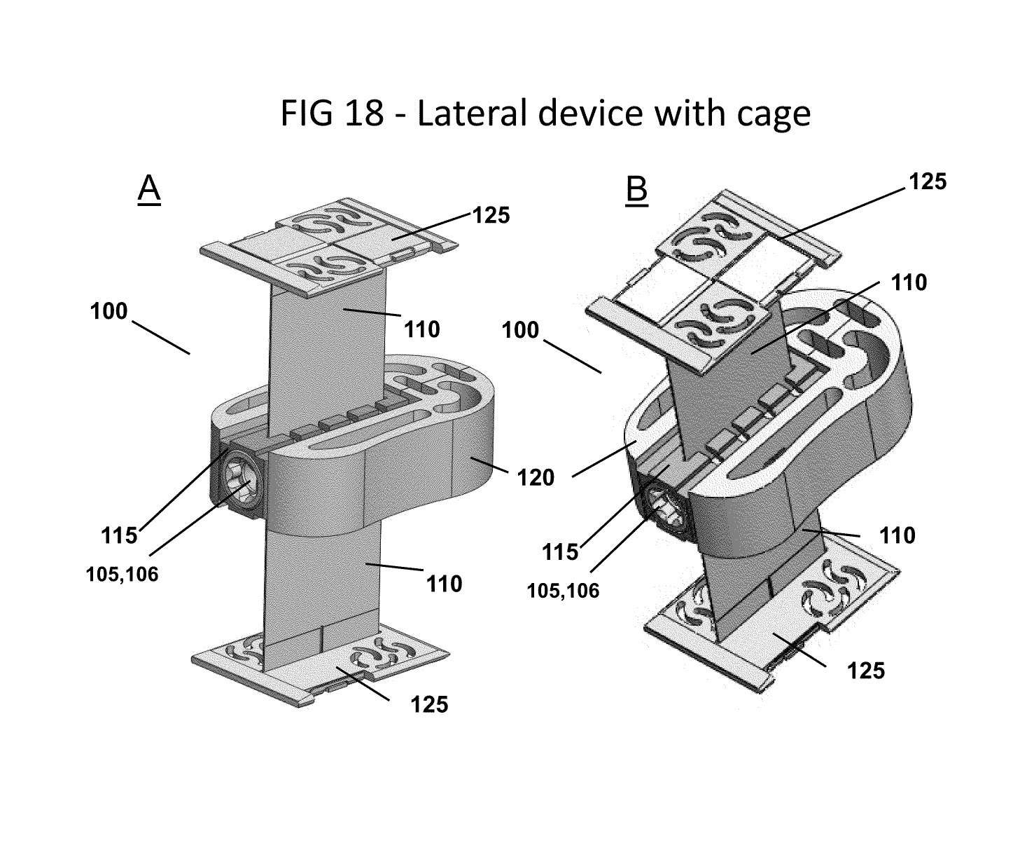

Voids within the cages 120 provide spaces into which fillers such as bone putty, bone paste, bone cement, grafted bone, mesenchymal stem cells, osteoblasts, depot-release drug or biopharmaceutical formulations (including, without limitation, bone morphogenic protein 2), can be placed. Alternatively, the voids which can remain open to permit tissue ingrowth. Other portions of the apparatus 100, particularly the vertebral securement features 125 as shown in FIGS. 17-18, can include voids either for the addition of materials such as those set forth above, or to permit tissue ingrowth.

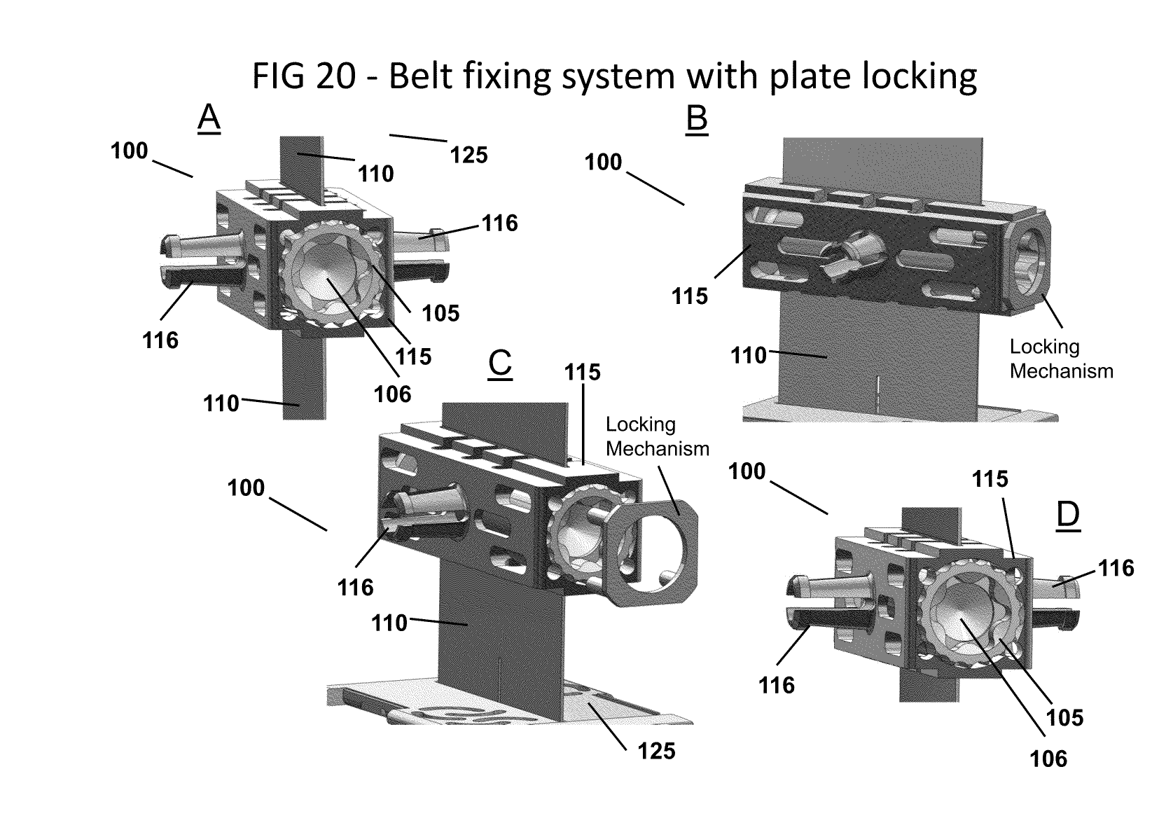

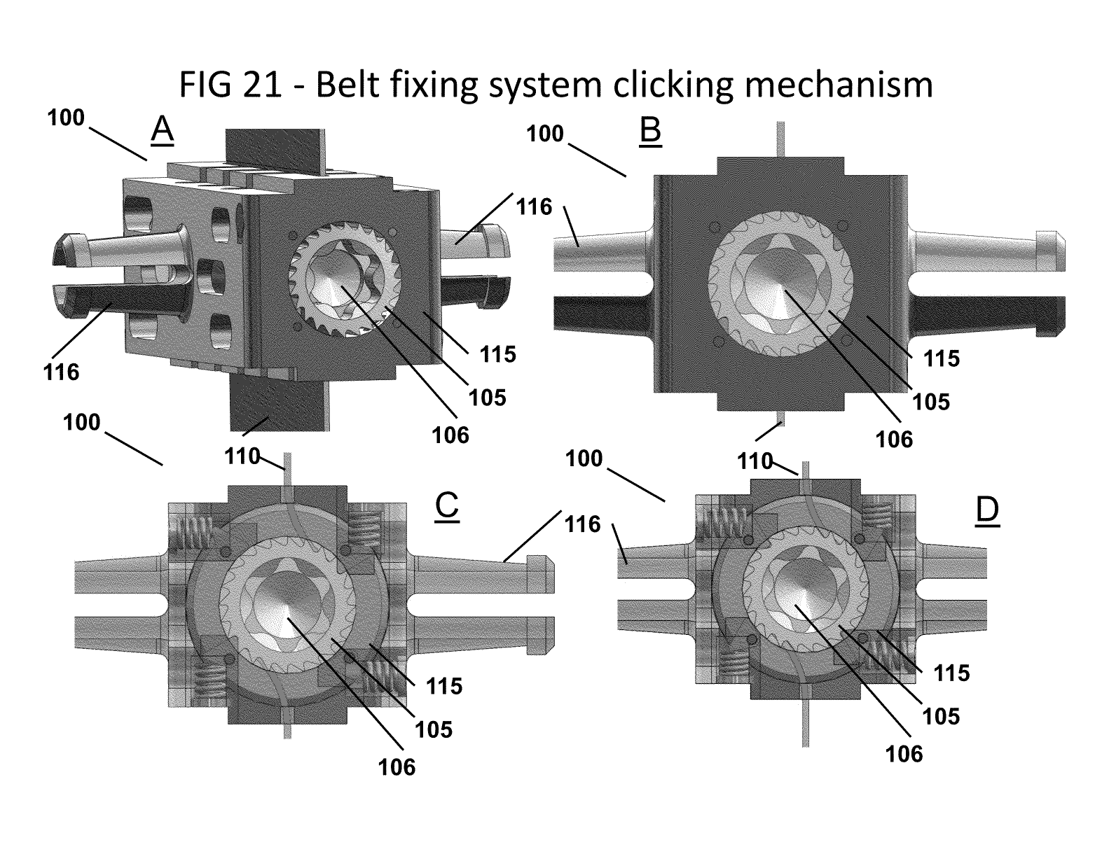

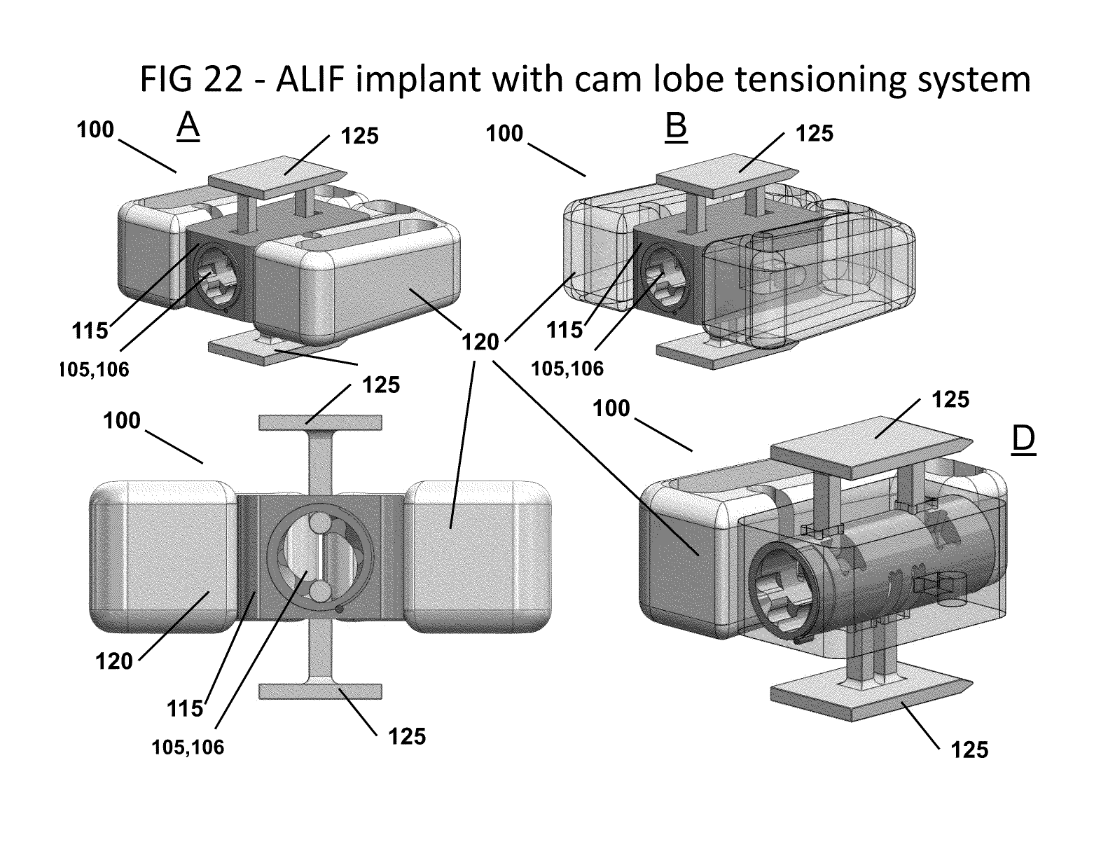

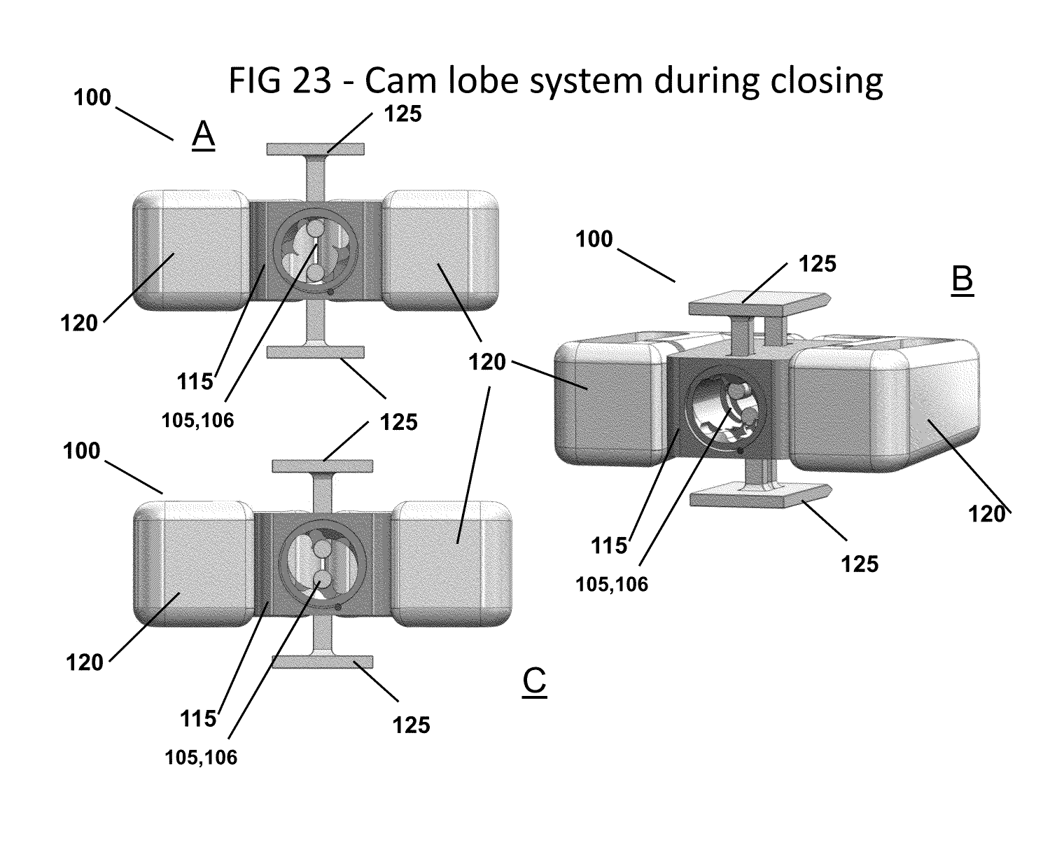

The housing 115 optionally includes a feature to lock the central rotating member 105 in position, such as a locking plate as shown in FIG. 20. Alternatively, the drive 106 can include a multi-toothed ring and one or more pawls 117, as shown in FIG. 21. While embodiments of the invention have been described with an emphasis on a single central rotating member 105 and a tape 110, other suitable arrangements will occur to those skilled in the art, such as the cam-lobe arrangement shown in FIGS. 22-23, as well as arrangements utilizing multiple rotating members, which may optionally be coupled to one another, etc.

Bioactive materials, including without limitation Bone Morphogenetic proteins such as BMP7, may be incorporated within or applied to surfaces of the appliance 100, for example to promote bone growth.

The termini of the tape 110 may be either flexible or rigid, and may incorporate strengthening elements that prevent deformation of the termini of the tape 110 and the vertebral securement features 125.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “bioactive material” as used herein should be understood to mean any substance that has a biological effect, including without limitation proteins and peptides, nucleic acids, lipids, carbohydrates, drugs, etc.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A medical device, comprising:
a housing;
a flexible tape passing through the housing, the flexible tape having first and second ends; and
an actuator disposed within the housing, the actuator contacting the flexible tape and configured to adjust a tension along a length of the flexible tape, thereby urging the first and second ends of the tape toward one another.

2. The medical device of claim 1, wherein each of the first and second ends includes a portion configured to anchor the end of the tape within a bone.

3. The medical device of claim 2, wherein the portion configured to anchor the end of the tape within a bone is substantially planar and includes a perforation, a bump, or a ridge.

4. The medical device of claim 1 wherein each of the first and second ends includes a strengthening element positioned to prevent deformation of the tape.

5. The medical device of claim 1, wherein (a) the actuator is a cylindrical body having an aperture through which the flexible tape passes, (b) the actuator is rotatably disposed within the housing and (c) the actuator includes a portion mateable with a tool to permit a user to rotate the actuator within the housing.

6. The medical device of claim 1, wherein the tape comprises a material selected from the group consisting of metal, plastic, polymer, mesh and woven material.

7. The medical device of claim 1, wherein the housing includes at least one flanged extension, the medical device further comprising a bone cage connected to the housing via the flanged extension.

8. A system for treating a patient, the system comprising:
the medical device of claim 1; and
a delivery device comprising a plurality of bone chisels defining a space sized to accommodate the medical device.

9. The system of claim 8, wherein the space is sized to separate the first and second ends by a distance sufficient to permit each of the first and second ends to be anchored within the cancellous bone of a first vertebra and a second vertebra such that the housing rests between the first and second vertebra.

10. A delivery device for a spinal fusion appliance, the delivery device comprising:
a plurality of bone chisels defining a space sized to accommodate the spinal fusion appliance.

11. The delivery device of claim 10, wherein the space is shaped to accommodate a spinal fusion appliance having a housing and a flexible tape extending through the housing.

12. The delivery device of claim 10 further comprising a handle and a shaft having a length sufficient to deliver the spinal fusion appliance to a site where spinal fusion is desired when the shaft is inserted through an abdominal incision and the handle remains outside of the body.

13. A method of treating a patient in need of interbody fixation, the method comprising the steps of:
providing a medical device comprising:
a housing;
a flexible tape passing through the housing, the flexible tape having first and second ends; and
an actuator disposed within the housing, the actuator contacting the flexible tape and configured to adjust a tension along a length of the flexible tape, thereby urging the first and second ends of the tape toward one another;
positioning the medical device in relation to first and second vertebrae such that the first and second ends of the flexible tape are positioned within the cancellous bone of the first and second vertebrae, respectively, and the housing is positioned between the first and second vertebrae; and
adjusting the actuator to urge the first and second ends of the tape toward one another, thus urging the first and second vertebrae toward the housing.

14. The method of claim 13, wherein positioning the medical device comprises inserting the medical device through an abdominal incision.

15. The method of claim 14, wherein the medical device is positioned using a delivery device, the delivery device comprising a plurality of bone chisels defining a space sized to accommodate the medical device.
